(19) United States
(12) Patent Application Publication
(10) Pub. No.: US 2024/0000986 A1
(43) Pub. Date: Jan. 4, 2024

(54) HEAT RETENTIVE DIFFUSIVE LIGHT WEIGHT ANTI-MICROBIAL STEAM NOZZLE ENABLED WITH A CONVERGENT ENVELOPE

(71) Applicant: MHI Health Devices, LLC., Cincinnati, OH (US)

(72) Inventors: Jainagesh Sekhar, Cincinnati, OH (US); Venkata Burada, Cincinnati, OH (US); Michael Connelly, Cincinnati, OH (US)

(73) Assignee: MHI Health Devices, LLC., Cincinnati, OH (US)

(21) Appl. No.: 18/197,792

(22) Filed: May 16, 2023

Related U.S. Application Data
(60) Provisional application No. 63/345,075, filed on May 24, 2022.

(51) Int. Cl. A61L 2/07

(52) U.S. Cl. CPC A61L 2/07

(57) ABSTRACT

Presented is an apparatus and a means to more broadly apply a projected fluid to a surface by the passing of the fluid through a convergent diffuser. The use of a convergent diffuser runs counter to the present state of the art which suggests the use of a divergently configured diffuser. A preferred embodiment teaches a construction of the diffuser employing mesh configurations in various shapes. Another embodiment envisions a diffuser configured of a coil of material formed in a donut shape through which a fluid passes and is dispersed.

### CROSS-REFERENCE TO RELATED APPLICATIONS

This application claims the benefit of U.S. provisional application 63/345,075 entitled “Heat Retentive Diffusive Light Weight Anti-microbial Steam Nozzle Enabled With a Convergent Envelope” filed on May 24, 2022, by the inventors, the disclosure of which is incorporated by reference herein in its entirety. This application also contains references to U.S. patent application Ser. No. 17/462,052, published as US2022/0072577 A1, entitled “Fluid Nozzle Guard” filed by the inventors on Sep. 31, 2021, which is disclosed herein by reference in its entirety.

### BACKGROUND

In many fluid application situations, an application device is needed capable of providing an expanded area of coverage. To expand the coverage of a sprayed fluid, a diffuser (expander) is often employed. However, for antibacterial treatment, the standard diffuser design may cause a loss of efficiency. A fluid nozzle providing optimal coverage to a surface or to provide the most effective application of a fluid is needed. Such a device could be used to optimally treat a surface rapidly that is bearing microbes yet prevent overheating or burning of the surface from heat guns or steaming devices.

The food industry requires a larger area of coverage regarding the impact of superheated steam and steam mixtures on affected surfaces. An extremely lightweight device is needed having a fluid nozzle that can disperse the superheated steam yet not lose effectiveness (superheated temperature for rapid microbial removal). Such a device could be used to quicken the pace of antibacterial cleaning. These devices may produce steam and steam mixtures that are significantly more potent than required by the FDA rule for 5 log reduction in 30 minutes.

### SUMMARY

Herein is described a diffuser attachment to a nozzle that can spread directed, effective applications of a fluid to increase the contact area of treatment and hence improve productivity and reduce human energy use. A new diffuser is proposed which converges down from the nozzle (exhaust end of the application device) but enables a diffusion type of action. This application teaches away from the standard common nozzles where an expander is required to cause diffusion (diffusion may be defined as the converting of a narrow dense stream of a fluid into a broader, gentler application). Also, expander nozzles are often heavy since they are built to increase the flow area, and consequently use more material than the original discharge tube or nozzle. This application presents a lightweight heat retentive diffuser nozzle that has a converging designed envelope rather than a diverging envelope design.

Contemplated fluid projection devices may include, but are not limited to, steam generators, heat guns, pressure washers, grit blasters, and paint sprayers. The nozzle of any of these devices may be equipped with a guard comprised with at least one spacing arm, possibly comprised of a coil spring, configured to a specified length, depending on the application, which will allow the exhaust end of the fluid projection device to get no closer than the length of the spacing arm of the guard. The length will be such that the fluid is applied in the “sweet spot” which optimizes coverage. The nozzle guard equipped with spring spacing arms, while allowing an approach of a set distance from a surface, will allow some lateral movement parallel to the working surface as the spring flexes and bends, but not enough to appreciably reduce the distance between the nozzle and the surface resulting in surface damage.

The mechanical attachment of the nozzle guard to, or near, the exhaust nozzle may be permanent or temporary. Temporary attachments may be adjustable. The nozzle guard may be welded or affixed with screws, rivets, or other fasteners to the nozzle. The base of the nozzle may be threaded allowing for a guard provided with a like threaded ring affixed to the spring to be screwed onto the nozzle. Clamping and friction fitting of the nozzle guard equipped with a non-threaded ring are also contemplated.

The length of the nozzle guard will depend on the fluid that that is projected, and the velocity and strength of the projection and coverage required. The nozzle guard will be configured of a material capable of resisting the fluid to be projected (heat, cold, wear, abrasion, etc.). In most cases, one spacing arm will be sufficient, but in others, more than one arm is contemplated, possibly positioned around the exhaust nozzle of the projection device.

### DRAWING—FIGURES

FIG. 1 is a representation of a nozzle guard for a superheated steam generation device or other fluid projection apparatus having dual spacing arms comprising coil springs.

FIG. 2 depicts a superheated steam generation device equipped with a nozzle guard at the exhaust end of the device.

FIG. 3 is a drawing demonstrating the difference between a converging nozzle and a diverging nozzle.

FIG. 4 is a close-up of nozzle guard equipped with a converging mesh diffuser affixed to a heat generation device.

FIG. 5 is a close-up of an embodiment of a converging mesh fluid diffuser.

FIG. 6 is a close-up of a further embodiment of a converging mesh fluid diffuser in a thimble shape.

FIG. 7 is an embodiment of fluid diffuser comprised of coiled wire or other material formed into a donut shape. Fluid would flow perpendicular to and through the presented donut shaped face.

FIG. 8 is a sideview of a nozzle having an outlet or exhaust comprising a tube with a coiled wire or other material formed into a donut shape placed partially within the nozzle at the end of the tube. The arrow indicates the direction of fluid flow.

FIG. 9 is a cut-away view of an exhaust tube with nozzle showing the positioning of multiple donut shaped diffusers placed along the path of a fluid flow. The arrow indicates the fluid flow direction.

FIG. 10 is a cut-away view a fluid exhaust perpendicular to the fluid flow.

DRAWING - REFERENCE NUMERALS

| | | | |
|---|---|---|---|
| 10 | fluid nozzle guard | 20 | spacing arm |
| 25 | cap | 30 | base |
| 35 | threads | 50 | superheated steam generator |
| 60 | nozzle | 70 | diffuser |
| 75 | donut diffuser | 80 | coil |
| 85 | nozzle tube | 90 | exhaust tube |
| 95 | sheet metal sleeve | 100 | spacer |

### DESCRIPTION

A preferred embodiment of the presented diffusive nozzle guard is for use with superheated steam generators; however, the use of the described guard and diffuser is contemplated with any fluid producing or projection device requiring a diffused spray or a greater area of fluid coverage. In this embodiment the guard would be heat retentive as well. A nozzle guard is contemplated with spacing arms, possibly comprised of springs positioned in line with a steam exhaust nozzle at a length determined to prevent damage to surfaces by the steam. Other embodiments may have more than one nozzle guard or spacing arms.

The nozzle guard is affixed to the steam generator next to the steam exhaust nozzle generally parallel to the nozzle and parallel to the direction of the generated steam. The spacing arm, or arms, may be equipped with a hard, plastic cap at the end intended to contact a surface. The purpose of the guard is to prevent a user from placing the device too close to soft matter such as PPE (cloth) or paper. The steam and steam gas velocity may experience flow velocities from a low 0.1 m/s to 10 m/s at the hot exit at about 100-1000° C. The guard is further comprised of a diffuser affixed to the nozzle at the point at, or near, where the nozzle guard is attached to the nozzle. The diffuser may be comprised of a metallic wire mesh. The mesh may be configured in various shapes and acts to spread out a thin fluid stream into a broader diameter of application. Also, by making the metallic mesh out of metal the heat can be manipulated and retained.

In this embodiment, the nozzle and nozzle guard need to be made of a material that withstands 500° C., allows lateral flexibility, does not corrode in steam or air, adheres to materials, and can be welded to an attachment that screws on to the application nozzle. The nozzle guard may be constructed of material of about 1/16″ in diameter and is 3″-6″ in length. It is comprised of a lightweight material that does not absorb the emitted heat of the steam but transmits the heat effectively. The nozzle guard may be configured allowing for fixturing of temperature indicators and biomarkers to enable a method of ensuring that that the steam impact region is at a point where a microbe load reduction efficacy is assured and optimized.

The nozzle guard may be configured at lengths to accommodate the heat resistances of various materials. The nozzle guard configuration will allow some lateral (sideways) movement along the surface perpendicular to the steam flow. Stiffness in the nozzle guard needs to be maintained to prevent it from bending and allowing the steam to get too close to the surface.

As stated above, the nozzle may be equipped with a means of diffusion that may be comprised of fine wire mesh having hole diameters from 0.01 mm to 3 mm and a corresponding web of the mesh of fine wire with an average diameter between 0.001 and 1 mm. Stainless steel and other heat resistant wire meshes are contemplated. The minimum distance between the exhaust port and a surface allowed by the guard will be such that the fluid is applied in a “sweet spot” where the fluid is most effectively applied to an optimized surface. When used with a superheated steam generator the wires of the diffuser mesh retain the heat as well as the steam in a gaseous state because the wire mesh is heated by the steam. The holes of the mesh diffuser also act to prevent ejection of any water. The mesh diffuser may be configured in any desired shape that retains the heat of the diffused fluid while dispersing it over a wide area including, but not limited to, convergent, conical, round or balloon shapes. The mesh diffuser will act to disperse a fluid flow (superheated steam) over a broader area than an undiffused stream. It has been found that mesh configurations that are convergent (taper down to a smaller diameter away from the nozzle) rather than divergent (become larger in diameter away from the nozzle and exhaust end of device) spread out the flow of superheated steam over a larger area in a more effective manner than the standard divergent design that is taught by the prior art. In practice, the fluid stream comes into contact with the mesh and is directed in the direction of the mesh, possibly perpendicular to, or at an angle away from, the mesh opening thereby dispersing the flow. Such diffusers may be affixed to the nozzle guard in a position close to the exhaust end or nozzle of the fluid projection device. Such convergent diffusers may also be used with nozzle not comprising guards.

### DETAILED DESCRIPTION

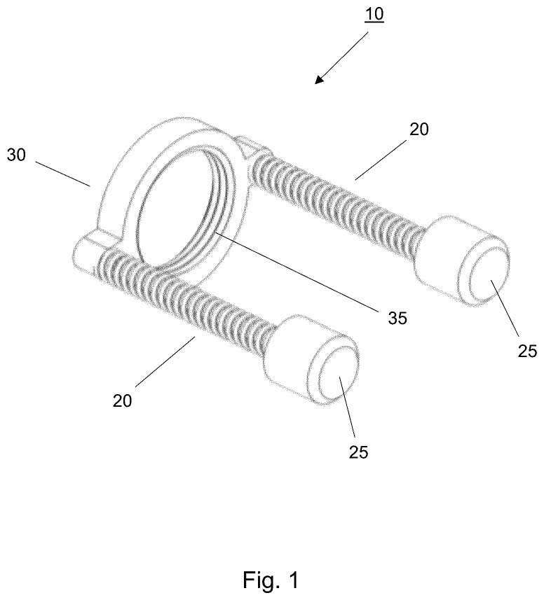

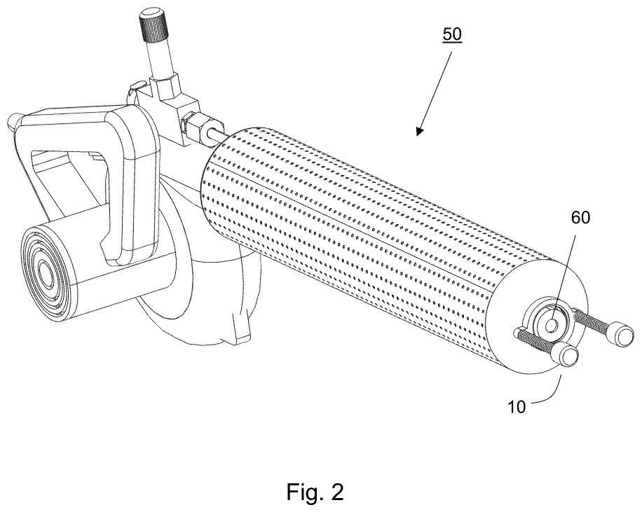

FIG. 1 is a representation of a fluid nozzle guard 10 to be attached to a fluid dispersion or generation device such as a superheated steam generator 50 as shown in FIG. 2. The guard 10 is comprised of a base 30, in this case having threads 35 to affix the base 30 to the exhaust end of a fluid generation device such as the steam generator 50 of FIG. 2. The nozzle guard 50 may be further comprised of spacing arms 20 extending away from the base 30 and may be equipped with caps 25 to protect any contacted work surfaces. In this embodiment the arms 20 are constructed of coil springs which keep the nozzle 60 and any projected fluid at a minimum distance from a surface. Other embodiments are described in U.S. patent application Ser. No. 17/462,052, published as US2022/0072577 A1, which is disclosed herein by reference in its entirety.

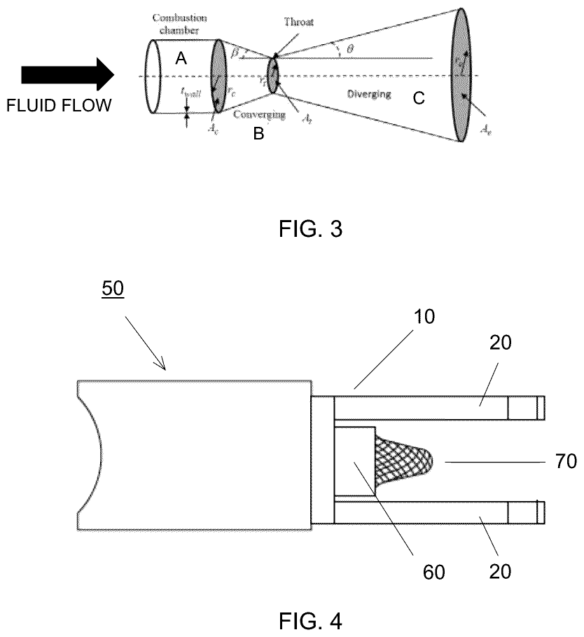

FIG. 3 is a representation of the difference between a convergent and a divergent orientation. The arrow to the left of the figure pointing right indicates the fluid flow direction, here, through a combustion chamber A. A converging shape B is to the right of the combustion chamber A. The converging shape is characterized by a conic shape having a larger diameter nearer to the source of the fluid flow than farther way from it. Conversely, the diverging shape C, farthest to the right, is a conic shape comprising a smaller diameter nearer to the source of the fluid flow and a larger diameter at its exhaust farther away from the fluid flow source.

FIGS. 4, 5 and 6 show contemplated embodiments of a fluid diffuser 70 affixed to a nozzle 60 in a roughly conical shape with the smaller diameter or tapered end positioned away from a superheated steam generator 50 or other fluid producing or distribution device. In a preferred embodiment, the diffuser 70 is constructed of a mesh material suitable for the specific application. In a steam or heat application mesh comprised of stainless steel, nickel alloy or other heat resistive material is contemplated. Other materials may be used that provide a non-stick, corrosive resistant or cleanable mesh for paint or chemical applications. Non-conductive materials are anticipated for applications in volatile environments.

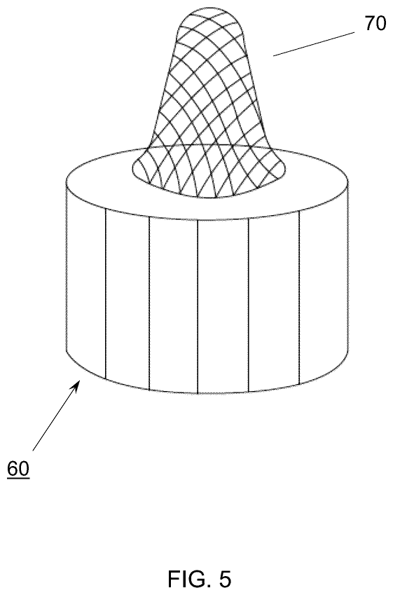

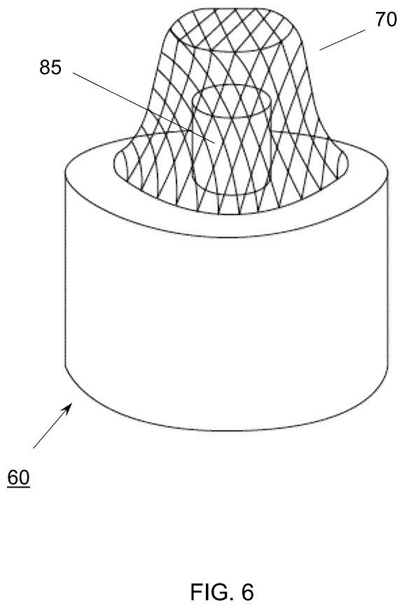

FIGS. 4 and 5 illustrate a diffuser 70 in a conical shape having a rounded tip away from the nozzle 60. Being conical the diffuser 70 has a larger diameter at the junction with the nozzle 60 gradually tapering to a smaller diameter at the rounded tip away from the nozzle 60 and source of the fluid and projection device. Various tapers and shapes of diffuser 70 tips are anticipated depending on the specific fluid and application. FIG. 6 depicts a diffuser 70 having a truncated cone or “thimble” shape with a flat tip.

FIG. 4 further shows the positioning of a diffuser 70 on a nozzle 60 within the spacing arms 20 attached to a cut-a-way view of a superheated steam generator 50. This converging orientation, along with other contemplated converging orientations, when comprised of a mesh material allows for a projected fluid to contact the mesh and be dispersed by it through the holes in the mesh material. The fluid is broadcast through the mesh in the direction of the individual spaces in the material. The fluid directly strikes the mech and its holes since it, and they, are in a direct line of the fluid flow. A diverging orientation, in contrast, would not broadcast the fluid in a similar manner as the fluid flow would not be in a direct line with the outward tapering conical surface.

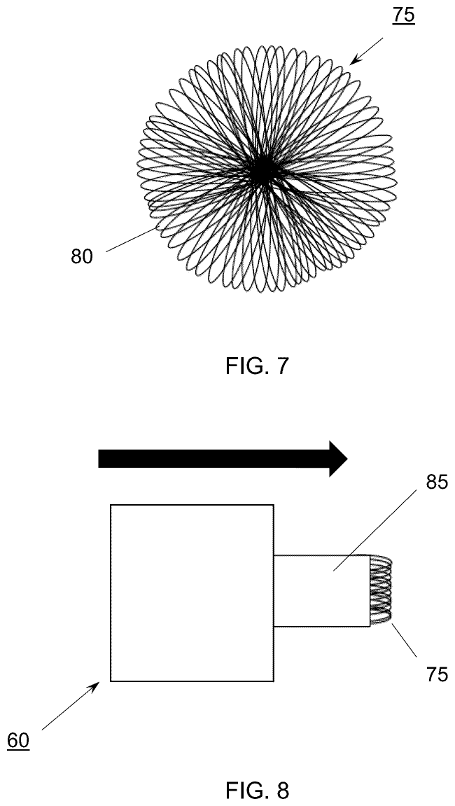

A further embodiment of the contemplated fluid diffuser is presented in FIGS. 7 and 8. FIG. 7 shows a donut diffuser 75 comprised of a coil 80 of straight wire stock formed into a donut or round shape. The coil 80 may be made of heat resistant metal wire or other appropriate material. A preferred donut diffuser 75 is simply constructed of a length of metal wire that is bent into a coil 80. The ends of the coil 80 of wire are then brought together forming a torus or donut configuration. The ends of the coil 80 may overlap and entwine together forming a coiled torus with more than one wire strand. Such entwining serves to hold the donut diffuser 75 together and creates more open spaces for fluid diffusion. The donut shaped face of the diffuser 75 would be formed at a right angle or perpendicularly to the coils 80 themselves. As with the aforementioned embodiments, this diffuser embodiment retains the heat of a heated fluid and helps to retain liquid. The donut of coiled wire may then be placed in the path of a projected fluid typical confined in a constricted pathway such as a nozzle, chamber or tube. It is generally anticipated that the diffuser would be placed in a manner where the fluid would contact and flow through the rounded or donut shaped face of the diffuser at a right angle. Other configurations are also anticipated.

FIG. 8 shows the donut diffuser 75 inserted into the end of a fluid nozzle tube 85 of nozzle 60. The expelled fluid would flow through the nozzle tube 85 and contact the donut shaped face of the diffuser at a right angle and be dispersed and diffused by the wires and openings of the coil. Such diffusers may be placed completely within a nozzle or other area of flow to provide diffusion and heat retention singly or in multiples depending on the application. In FIG. 8 fluid flow direction is indicated by the arrow.

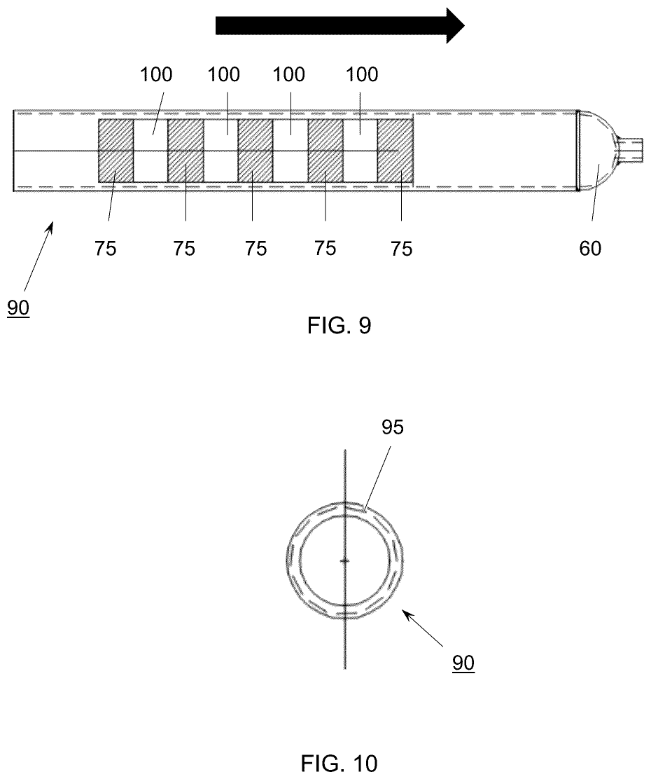

An embodiment of an exhaust tube 90, or constricted space, with an affixed nozzle 60 that may be part of a superheated steam generator 50 or other fluid application device is depicted in cross section along the fluid flow as indicated by the arrow in FIG. 9. FIG. 10 shows a cross section of the exhaust tube 90 perpendicular to the fluid flow. The exhaust tube 90 is round in cross-section and may by partially or completely within the casing or shroud of a fluid projection device. The exhaust tube 90 comprises a hollow chamber containing multiple donut diffusers 75 positioned with their round faces perpendicular to the fluid flow. The diffusers may be separated by spacers 100 comprised of ceramic, air or other materials depending on the fluid and application. In heat generation applications the diffusers 75 may act to disperse the heated fluid or retain the heat of it. A thin sheet metal sleeve 100 may be placed lining the inner wall of the exhaust tube 90 and nozzle 60 to provide extra heat retention. It is contemplated that the diffusers 75 and the sheet metal sleeve 100 may be energized by electricity or other means up to at least 75% of the exit gas to provide further heat.

Although preferred embodiments of the fluid diffuser and method are presented in the above specification, the scope of the invention is not to be limited by them. Other diffuser configurations and fluid applications and equivalents are anticipated by the applicants.

We claim:

1. A fluid diffuser for use with a fluid projection and application device configured in a generally convergent orientation away from an exhaust nozzle of the fluid projection device wherein a fluid is projected through the diffuser, whereby the diffuser provides a broader area of coverage of the fluid.

2. The diffuser of claim 1 wherein the diffuser is comprised of a mesh and wherein the mesh is configured in an envelope having sides and a top thereby encasing the exhaust nozzle creating a barrier through which the fluid passes.

3. The diffuser of claim 2 wherein the mesh is comprised of stainless steel.

4. The fluid diffuser of claim 2 having a conical shape.

5. The fluid diffuser of claim 4 wherein the conical shape is truncated.

6. The fluid diffuser of claim 2 having a rounded balloon shape.

7. A method for the diffusion of a projected fluid comprising projecting the fluid through a fluid diffuser wherein the diffuser is configured in a generally convergent orientation away from an exhaust nozzle of the fluid projection device wherein a fluid is projected through the diffuser, whereby the diffuser provides a broader area of coverage of the fluid.

8. The method of claim 7 wherein the fluid diffuser is comprised of a mesh and wherein the mesh is configured in an envelope having sides and a top thereby encasing the exhaust nozzle creating a barrier through which the fluid passes.

9. The fluid diffuser of claim 8 having a conical shape.

10. The fluid diffuser of claim 8 wherein the conical shape is truncated.

11. A fluid diffuser for use along the flow of a projected fluid through which the fluid passes inside a constricted space comprising at least one coil of material having two ends wherein the ends are positioned with the ends overlapping forming a face perpendicular to the coils in a donut shape allowing for fluid improved dispersion and heat retention of the fluid.

12. The fluid diffuser of claim 11 wherein the at least one coil of material is positioned at the output end of the constricted space.

13. The fluid diffuser of claim 11 wherein the at least one coil of material is positioned within the constricted space away from the output.

14. The fluid diffuser of claim 11 wherein the at least one coil of material is comprised of wire.

15. The fluid diffuser of claim 14 wherein the wire is comprised of metal.

16. The fluid diffuser of claim 11 wherein the coils in a donut shape are positioned in a manner allowing the fluid to pass perpendicularly through the donut shape.

17. The fluid diffuser of claim 11 wherein the constricted space is comprised of a sheet metal sleeve.

18. The fluid diffuser of claim 17 wherein the metal sleeve and is energized.

19. The fluid diffuser of claim 11 wherein the at least one coil of material is energized.
